(19) United States
(12) Patent Application Publication
(10) Pub. No.: US 2013/0296430 A1
(43) Pub. Date: Nov. 7, 2013

(54) COMPOSITIONS AND METHODS FOR TREATING AUTISM AND AUTISM SPECTRUM DISORDER

(71) Applicants: Antonio Hardan, Palo Alto, CA (US); Rabin Tirouvanziam, Redwood City, CA (US); Leonora A. Herzenberg, Stanford, CA (US)

(72) Inventors: Antonio Hardan, Palo Alto, CA (US); Rabin Tirouvanziam, Redwood City, CA (US); Leonora A. Herzenberg, Stanford, CA (US)

(21) Appl. No.: 13/792,361

(22) Filed: Mar. 11, 2013

Related U.S. Application Data
(60) Provisional application No. 61/642,156, filed on May 3, 2012.

(51) Int. Cl. A61K 31/198; A61K 45/06

(52) U.S. Cl. CPC A61K 31/198; A61K 45/06
USPC 514/562

(57) ABSTRACT

The described invention provides a method for treating a behavioral deficit, such as irritability and stereotypic/repetitive behaviors, in a subject with autism spectrum disorder by administering a composition comprising a therapeutic amount of N-acetylcysteine, a derivative of N-acetylcysteine, or a pharmaceutically acceptable salt of N-acetylcysteine.

### CROSS-REFERENCE TO RELATED APPLICATIONS

This application claims priority from U.S. provisional patent application Ser. No. 61/642,156, filed May 3, 2012, the entire disclosure of which is incorporated herein by reference.

### FIELD OF INVENTION

The described invention is related to compositions and methods for treating autism and autistic spectrum disorders.

### BACKGROUND

### 1. Autism

Autism is the most severe and devastating condition in the broad spectrum of developmental disorders called “pervasive developmental disorders” (Rapin, I., New Engl. J. Med., 337: 97-104 (1997)). Autistic disorders are characterized by marked impairment in communication and reciprocal social interaction, social skills, verbal communication, behavior, and cognitive function (Rapin, I. New Engl. J. Med., 337: 97-104 (1997); Lord, C. et al., Neuron, 28, 355-363 (2000)). Abnormalities in language development, mental retardation, and epilepsy are frequent problems in the clinical profile of patients with autism. The core symptoms of autism include abnormal communication, social relatedness, behavior, and cognition (Rapin, I., N. Engl. J. Med., 337: 97-104 (1997) and Lord, C. et al., Neuron, 28, 355-363 (2000))

Causes of autism remain elusive yet are thought to be the culmination of genetic, developmental, and environmental factors. In most patients, the cause(s) are still unknown (Rapin, I., and Katzman R., Annals of Neurology, 43, 7-14 (1998); Newschaffer, C. et al., Epidemiology Reviews, 24, 137-153 (2002); Cohen, D. et al., Journal of Autism & Developmental Disorders, 35, 103-116 (2005)).

### 1.1. Neurobiology of Autism

### 1.2. Clinical and Epidemiological Aspects of Autism

Although the neurobiological basis for autism remains poorly understood, several lines of research now support the view that genetic, environmental, neurological, and immunological factors contribute to its development (Rapin, I., and Katzman R., Annals of Neurology, 43, 7-14 (1998); Newschaffer, C. et al., Epidemiology Reviews, 24, 137-153 (2002); Folstein, S. and Rosen-Sheidley, B., Nature Reviews Genetics, 2, 943-955 (2001); Korvatska, E., et al., Neurobiology of Disease, 9, 107-125 (2002)). Studies have suggested that several different genetic factors and/or other risk factors may be combined during development to produce complex changes in Central Nervous System (CNS) organization, which translate into abnormalities of neuronal and cortical cytoarchitecture responsible for the complex language and behavioral problems that characterize the autistic phenotype.

The majority of autistic children show abnormalities during infant development that may not become apparent until the second year of life. Approximately 30-50% of children undergo regression, with a loss of skills, including language, between 16 and 25 months of age. In the medical evaluation of autism, specific etiologies can be found in <10% of children, including fragile X, tuberous sclerosis, and other rare diseases (Cohen, D. et al., Journal of Autism & Developmental Disorders, 35, 103-116 (2005)). Epilepsy occurs in up to 40% of patients, and epileptic discharges may occur on EEGs early in childhood, even in the absence of clinical seizures (Tuchman, R. and Rapin, I., Lancet Neurology, 1, 352-358 (2002)). Although children with autism present with a wide spectrum of symptoms that vary in severity and clinical progression, it is possible to define these features in affected individuals and follow them over time (Aman, M. et al., CNS Spectrums, 9, 36-47 (2004)).

### 1.3. Neuroanatomical Abnormalities in Autism

A wide range of anatomical and structural brain abnormalities have been observed in autistic patients by longitudinal clinical and magnetic resonance imaging studies. The clinical onset of autism appears to be preceded by two phases of abnormalities in brain growth: a reduced head size at birth and a sudden and excessive increase in head size between 1-2 months and 6-14 months (Courchesne et al., Curr. Opin. Neurol., 17, 489-496 (2004)). These studies have also shown that the most abnormal pattern of brain overgrowth occurs in areas of the frontal lobe, cerebellum, and limbic structures between 2-4 years of age, a pattern that is followed by abnormal slowness and an arrest in brain growth (Courchesne et al., Curr. Opin. Neurol., 17, 489-496 (2004); Courchesne, E. and Pierce, K., International Journal of Developmental Neuroscience, 23, 153-170 (2005)). Other studies of high-functioning autistic patients have shown an overall enlargement of brain volume associated with increased cerebral white matter and decrease in cerebral cortex and hippocampal-amygdala volumes (Herbert et al., Brain, 126, 1182-1192 (2003); Herbert et al., Annals of Neurology, 55, 530-540 (2004)). However, the cause of this dissociation and of these patterns of abnormal brain growth is not understood.

Other studies have shown that disruption of white matter tracts and disconnection between brain regions are present in autistic patients, as demonstrated by, e.g., diffusion tensor imaging. This approach has demonstrated reduced fractional anisotropy values in white matter adjacent to the ventromedial prefrontal cortices, anterior cingulate gyms, and superior temporal regions, findings suggestive of the disruption in white matter tracts in brain regions involved in social functioning that has been described in autistic patients (Barnea-Goraly et al., Biological Psychiatry, 55, 323-326 (2004)).

In addition to abnormal growth patterns of the brain, one of the most consistent findings of neuroimaging studies in autism is the presence of abnormalities in the cerebellum. Reduction in the size of cerebellar regions such as the vermis (Hashimoto et al., Journal of Autism & Developmental Disorders, 25, 1-18 (1995); Kaufmann et al., Journal of Child Neurology, 18, 463-470 (2003)), an increase in white matter volume, and reduction in the gray/white matter ratio (Courchesne, E. and Pierce, K., International Journal of Developmental Neuroscience, 23, 153-170 (2005)) are the most prominent changes observed in the cerebellum. In one of these studies, the cerebellar changes appeared to be specific to autism, in contrast to other neurodevelopmental disorders such as Down syndrome, Down syndrome with autism, fragile X and fragile X with autism (Kaufmann et al., Journal of Child Neurology, 18, 463-470 (2003)). These observations concur with: (1) the findings from neuropathological studies describing abnormalities in the cerebellum, such as a decreased number of Purkinje cells (Kemper, T. and Bauman, M., Journal of Neuropathology & Experimental Neurology, 57, 645-652 (1998); Bailey et al., Brain, 121(Pt 5), 889-905 (1998)) and, most recently, (2) observation of increased microglial activation and astroglial reactions in both the granular cell and white matter layers and a reduction in Purkinje and granular cells (Vargas et al., Annals of Neurology, 57, 67-81 (2005)).

### 1.4. Neuropathology of Autism

Cytoarchitectural organizational abnormalities of the cerebral cortex, cerebellum, and other subcortical structures are the most prominent neuropathological changes in autism (Kemper, T. and Bauman, M., Journal of Neuropathology & Experimental Neurology, 57, 645-652 (1998); Bailey et al., Archives of Pediatric & Adolescent Medicine, 159, 37-44 (1998)). An unusual laminar cytoarchitecture with packed small neurons has been described in classical neuropathological studies, but no abnormalities in the external configuration of the cerebral cortex were noted (Kemper, T. and Bauman, M., Journal of Neuropathology & Experimental Neurology, 57, 645-652 (1998)). Cerebellar and brainstem pathology was prominent, with a loss and atrophy of Purkinje cells, predominantly in the posterior lateral neocerebellar cortex.

At least three different types of pathological abnormalities have been delineated in autism: (1) a curtailment of the normal development of neurons in the forebrain limbic system; (2) an apparent decrease in the cerebellar Purkinje cell population; and (3) age related changes in neuronal size and number in the nucleus of the diagonal band of Broca, the cerebellar nuclei, and the inferior olive (Kemper, T. and Bauman, M., Journal of Neuropathology & Experimental Neurology, 57, 645-652 (1998)). These observations suggest that delays in neuronal maturation are an important component in the spectrum. In addition to these cytoarchitectural abnormalities, the number of cortical minicolumns, the narrow chain of neurons that extend vertically across layers 2-6 to form anatomical and functional units, appeared to be more numerous, smaller, and less compact in their cellular configuration in the frontal and temporal regions of the brain of autistic patients compared with controls (Casanova et al., Neurology, 58, 428-432 (2002)). Pathological evidence of immunological reactions within the CNS, such as lymphocyte infiltration and microglial nodules, also has been described in a few case reports (Bailey et al., 1998, Brain, 121(Pt 5), 889-905; Guerin et al., Developmental Medicine & Child Neurology, 38, 203-211 (1996)).

### 1.5. Immunological Abnormalities in Autism

Reports of differences in systemic immune findings over the past 30 years have led to speculation that autism may represent, in some patients, an immune mediated or autoimmune disorder (Ashwood, P. and Van de Water, J., Autoimmunity Reviews, 3, 557-562 (2004)). Recent studies of immune dysfunction in autism have sought to understand these findings in the clinical context of the syndrome (Korvatska et al., Neurobiology of Disease, 9, 107-125 (2002); Ashwood, P. and Van de Water, J., Autoimmunity Reviews, 3, 557-562 (2004); Zimmerman, The immune system. In M. Bauman & T. L. Kemper (Eds.), The Neurobiology of Autism pp. 371-386, The Johns Hopkins University Press (2005)). Abnormalities of both humoral and cellular immune functions have been described in small studies of children with autism (N=10-36), and include decreased production of immunoglobulins or B and T-cell dysfunction (Warren et al., Journal of Autism & Developmental Disorders, 16, 189-197 (1986)). Early studies suggested that prenatal viral infections might damage the immature immune system and induce viral tolerance (Stubbs, E. and Crawford, M., Journal of Autism & Child Schizophrenia, 7, 49-55 (1977)), while later studies showed altered T-cell subsets and activation, consistent with the possibility of an autoimmune pathogenesis (Gupta et al., Journal of Neuroimmunology, 85, 106-109 (1998)). Recently, earlier reports of a four-fold increase in the serum complement (C4B) null allele (i.e., no protein produced) was confirmed in 85 children with autism, compared to controls.

Studies of peripheral blood have shown a range of abnormalities, including T-cell, B-cell, and NK-cell dysfunction; autoantibody production; and increased pro-inflammatory cytokines (Gupta et al., Journal of Neuroimmunology, 85, 106-109 (1998); Singh et al., Pediatric Neurology, 17, 88-90 (1997); Singh et al., Journal of Biomedical Science, 9, 359-364 (2002); Vojdani et al., Journal of Neuroimmunology, 129, 168-177 (2002); Jyonouchi et al., Journal of Neuroimmunology, 120, 170-179 (2001)). Shifts observed in Th1 to Th2 lymphocyte subsets and cytokines and associations with human leukocyte antigen (HLA)-DR4 have suggested the possibility that autoimmunity against brain antigens may contribute to the neuropathology of autism (van Gent et al., Journal of Child Psychology & Psychiatry, 38, 337-349 (1997)). Decreases in immunoglobulin subsets and complement, the presence of auto-antibodies against CNS antigens, and an effect of maternal antibodies have also been proposed as pathogenic factors (Dalton et al., Annal of Neurology, 53, 533-537 (2003)). In most of these studies, phenotyping was limited to descriptions of the subjects as “autistic” based on criteria of the Diagnostic and Statistical Manual of the American Psychiatric Association. “Abnormal” immune findings varied from 15-60% of children with autism. For some parameters, unaffected siblings showed intermediate values, and a background of such “abnormalities” was noted in normal controls as well. In all studies, measurements have been reported at single time points and among subjects of different ages. Since these differences in systemic immune findings in autism have not been followed in the same patients over time, it is not clear whether they reflect true immune dysfunction or represent dysmaturation that changes with age (Zimmermann, The Neurobiology of Autism, pp. 371-386, The Johns Hopkins University Press (2005)). Also, no clinical immunodeficiency states have been reported in association with unusual infections or reactions to immunizations, despite widespread interest in the possibility of such relationships (Halsey, N. and Hyman, S., Pediatrics, 107, E84 (2001)).

### 1.6. Immune-to-Brain Communication Pathways

The brain has long been considered an “immune-privileged” organ but this immune status is far from absolute and varies with age and brain region. Moreover, the brain contains immune cells, such as macrophages and dendritic cells, which are present in the choroid plexus and meninges. Brain parenchymal macrophages, known as microglial cells, are more quiescent than other tissue macrophages but can respond to inflammatory stimuli by producing pro-inflammatory cytokines and prostaglandins. In addition, both neuronal and non-neuronal brain cells express receptors for these mediators (Dantzer et al., Nat Rev Neurosci., 9: 46-56 (2008)).

The brain monitors peripheral innate immune responses by several means that act in parallel. One pathway involves afferent nerves: locally produced cytokines activate primary afferent nerves, such as the vagal nerves during abdominal and visceral infections and the trigeminal nerves during oro-lingual infections. In a second, humoral pathway, Toll-like receptors (TLRs) on macrophage-like cells residing in the circumventricular organs and the choroid plexus respond to circulating pathogen-associated molecular patterns by producing pro-inflammatory cytokines. As the circumventricular organs lie outside the blood-brain barrier, these cytokines can enter the brain by volume diffusion. A third pathway comprises cytokine transporters at the blood-brain barrier. Pro-inflammatory cytokines overflowing in the systemic circulation can gain access to the brain through these saturable transport systems. A fourth pathway involves IL-1 receptors that are located on perivascular macrophages and endothelial cells of brain venules. Activation of these IL-1 receptors by circulating cytokines results in the local production of prostaglandin E2.

Engagement of these immune-to-brain communication pathways ultimately leads to the production of pro-inflammatory cytokines by microglial cells. This process requires the convergent action of two events with different time courses: the activation of the rapid afferent neural pathway, and a slower propagation of the cytokine message within the brain. Activation of the neural pathway probably sensitizes target brain structures for the production and action of cytokines that propagate from the circumventricular organs and the choroid plexus into the brain. This way, the brain forms an “image” of the peripheral innate immune response that is similar in its elementary molecular components to the response in the periphery. The main difference is that this brain image does not involve an invasion of immune cells into the parenchyma and is not distorted by tissue damage that occurs at the site of infection.

The brain circuitry that mediates the various behavioral actions of cytokines remains elusive. The social withdrawal that characterizes cytokine-induced sickness behavior is unlikely to be mediated by the same brain areas as those underlying other responses to infection, such as reduced food consumption or activation of the hypothalamus-pituitary-adrenal axis. Ultimately, the site of action of the cytokine message depends on the localization of cytokine receptors or receptors for intermediates such as prostaglandins E2. These cytokine receptors are difficult to visualize on membranes because the number of receptor sites per cell is very low and they are easily internalized.

Nevertheless, IL-1 receptors were first localized in the granule cell layer of the dentate gyrus, the pyramidal cell layer of the hippocampus and the anterior pituitary gland. More recently, they were identified in endothelial cells of brain venules throughout the brain, at a high density in the preoptic and supraoptic areas of the hypothalamus and the sub-formical organ, and a lower density in the paraventricular hypothalamus, cortex, nucleus of the solitary tract and ventrolateral medulla.

### 1.7. Cytokine Profile in the Brain

Cytokines and chemokines play important roles as mediators of inflammatory reactions in the central nervous system (CNS) and in the process of neuronal-neuroglial interactions that modulate the neuroimmune system. Cytokines may contribute to neuroinflammation as mediators of pro-inflammatory or anti-inflammatory responses within the CNS. Recent studies have been focused on characterizing the profiles of cytokines and chemokines in autistic brains by assessing the relative expression of these proteins in tissue homogenates from medial frontal gyrus, anterior cingulate gyrus, and cerebellum of autistic and control patients by using cytokine protein array methodology. A statistical analysis of the relative expression of cytokines in autistic and control tissues showed a consistent and significantly higher level of subsets of cytokines in the brains of autistic patients. In particular, a larger spectrum of increases in pro-inflammatory and modulatory cytokines was seen in the anterior cingulate gyrus, an important cortical structure in autism, where there was a significant increase in pro-inflammatory cytokines such as interleukin-6 (IL-6), interleukin-10 (IL-10), macrophage chemoattractant protein-3 (MCP-3), eotaxin, eotaxin 2, macrophage-derived chemokine (MDC), chemokine-β8 (Ckβ8.1), neutrophil activating peptide-2 (NAP-2), monokine induced by interferon-γ (MIG) and B-lymphocyte chemoattractant (BLC) (Pardo C. et al., International Review of Psychiatry, 17: 485-495 (2005)).

The presence of macrophage chemoattractant protein-1 (MCP-1) is of particular interest, since it facilitates the infiltration and accumulation of monocytes and macrophages in inflammatory CNS disease (Mahad, D. and Ransohoff, R., Seminars in Immunology, 15: 23-32 (2003)). MCP-1 is produced by activated and reactive astrocytes, a finding that suggests the effector role of these cells in the disease process in autism. Studies have suggested that the increase in MCP-1 expression has relevance to the pathogenesis of autism as its elevation in the brain can be linked to pathways of microglial activation and perhaps to the recruitment of monocytes/macrophages to areas of neuronal/cortical abnormalities.

The presence of increased Transforming Growth Factor-β1 (TGF-β1) in the cortex and cerebellum of autistic brains may have important implications for the neurobiology of autism. Transforming Growth Factor-β1 (TGF-β1) is a key anti-inflammatory cytokine and is involved in tissue remodeling following injury. It can suppress specific immune responses by inhibiting T-cell proliferation and maturation and downregulates MHC class II expression (Letterio, J. and Roberts, A., Annual Review of Immunology, 16, 137-161 (1998)). Cells undergoing cell death have been shown to secrete TGF-β1, possibly to reduce local inflammation and prevent degeneration of additional surrounding cells (Chen et al., Immunity, 14, 715-725 (2001)). TGF-β1 is produced mostly by reactive astrocytes and neurons.

The elevation of TGF-β1 in autistic brains suggests that the elevation of this cytokine in autism may reflect an attempt to modulate neuroinflammation or remodel and repair injured tissue. A profile of cytokine up-regulation was observed in the anterior cingulate gyrus, a region in which several cytokines, chemokines, and growth factors were elevated markedly when compared to controls. Pro-inflammatory cytokines (e.g., IL-6) and anti-inflammatory cytokines (e.g., IL-10) as well as subsets of chemokines were elevated in the anterior cingulate gyrus, an important cortical region involved in dysfunctional brain activity in autism. These findings lent support to the conclusion that an active, ongoing immunological process was present in multiple areas of the brain but at different levels of expression in each area.

### 2. Autism Spectrum Disorder

Autism Spectrum disorders (ASD) are a heterogeneous group of neurodevelopmental disorders that manifest during early childhood and are characterized by stereotyped interests and impairments in social interaction and communication (American Psychiatric Association, Diagnostic and Statistical Manual of Mental Disorders DSM-IV-TR, 4^{th} ed. American Psychiatric Association Publishing Inc, Washington D.C. (2000)). Recent epidemiological studies have suggested that ASD is diagnosed in approximately 1% of children (Kogan et al., Pediatrics, 124(5): 1395-1403 (2009)), yet, little is known about the etiology and underlying neuropathology, and there are no clear biological markers for these disorders.

Recent studies show that immune dysfunction has been observed in many individuals with ASD, including, marked activation of microglia and increased levels of pro-inflammatory cytokines in brain tissue (Ashwood, P. et al., J. Neuroimmunol. 204 (1-2), 149-153 (2008); Enstrom, A. et al., Brain Behav. Immun. 24(1): 64-71 (2010)).

### 3. Free Radicals and N-Acetylcysteine (NAC)

A free radical is a highly reactive and usually short-lived molecular fragment with one or more unpaired electrons. Free radicals are highly chemically reactive molecules. Because a free radical needs to extract a second electron from a neighboring molecule to pair its single electron, it often reacts with other molecules, which initiates the formation of many more free radical species in a self-propagating chain reaction. This ability to be self-propagating makes free radicals highly toxic to living organisms.

Living systems under normal conditions produce the vast majority of free radicals and free radical intermediates. They handle free radicals formed by the breakdown of compounds through the process of metabolism. Most reactive oxygen species come from endogenous sources as by-products of normal and essential metabolic reactions, such as energy generation from mitochondria or the detoxification reactions involving the liver cytochrome P-450 enzyme system. The major cellular sources of free radicals under normal physiological conditions are the mitochondria and inflammatory cells, such as granulocytes, macrophages, and some T-lymphocytes, which produce active species of oxygen via the nicotinamide adenine nucleotide oxidase (NADPH oxidase) system, as part of the body's defense against bacterial, fungal, or viral infections. The major sources of free radicals, such as O₂⁻ and HNO₂⁻, are modest leakages from the electron transport chains of mitochondria, and endoplasmic reticulum.

Reactive oxygen species (“ROS”), such as free radicals and peroxides, represent a class of molecules that are derived from the metabolism of oxygen and exist inherently in all aerobic organisms. The term “oxygen radicals” as used herein refers to any oxygen species that carries an unpaired electron (except free oxygen). The transfer of electrons to oxygen also can lead to the production of toxic free radical species. The best documented of these is the superoxide radical. Oxygen radicals, such as the hydroxyl radical (OH⁻) and the superoxide ion (O₂⁻), are very powerful oxidizing agents and cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, can be converted by biological systems into other more reactive species, such as peroxyl (ROO⁻), alkoxyl (RO⁻), and hydroxyl (OH⁻) radicals.

Oxidative injury can lead to widespread biochemical damage within the cell. The molecular mechanisms responsible for this damage are complex. For example, free radicals can damage intracellular macromolecules, such as nucleic acids (e.g., DNA and RNA), proteins, and lipids. Free radical damage to cellular proteins can lead to loss of enzymatic function and cell death. Free radical damage to DNA can cause problems in replication or transcription, leading to cell death or uncontrolled cell growth. Free radical damage to cell membrane lipids can cause the damaged membranes to lose their ability to transport oxygen, nutrients or water to cells.

Biological systems protect themselves against the damaging effects of activated species by several means. These include free radical scavengers and chain reaction terminators; “solid-state” defenses, and enzymes, such as superoxide dismutase, catalase, and the glutathione peroxidase system.

Free radical scavengers/chemical antioxidants, such as vitamin C and vitamin E, counteract and minimize free radical damage by donating or providing unpaired electrons to a free radical and converting it to a nonradical form. Such reducing compounds can terminate radical chain reactions and reduce hydroperoxides and epoxides to less reactive derivatives.

Enzymatic defenses against active free radical species include superoxide dismutase, catalases, and the glutathione reductase/peroxidase system. Superoxide dismutase (SOD) is an enzyme that destroys superoxide radicals. Catalase, a heme-based enzyme which catalyzes the breakdown of hydrogen peroxide into oxygen and water, is found in all living cells, especially in the peroxisomes, which, in animal cells, are involved in the oxidation of fatty acids and the synthesis of cholesterol and bile acids. Hydrogen peroxide is a byproduct of fatty acid oxidation and is produced by white blood cells to kill bacteria.

Glutathione (GSH), a tripeptide composed of glycine, glutamic acid, and cysteine that contains a nucleophilic thiol group, is widely distributed in animal and plant tissues. It exists in both the reduced thiol form (GSH) and the oxidized disulfide form (GSSG). In its reduced GSH form, glutathione acts as a substrate for the enzymes GSH-S-transferase and GSH peroxidase, both of which catalyze reactions for the detoxification of xenobiotic compounds, and for the antioxidation of reactive oxygen species and other free radicals. Glutathione detoxifies many highly reactive intermediates produced by cytochrome P450 enzymes in phase I metabolism. Without adequate GSH, the reactive toxic metabolites produced by cytochrome P-450 enzymes may accumulate causing organ damage.

GSH plays key roles in cellular metabolism and protection against oxidative and other toxic molecules, including those generated in response to attack by cytokines that induce pain and fever. Stores of reduced GSH are influenced greatly by nutritional status, presence of certain disease states, and exposures to oxidative stressors and molecules that are detoxified by conjugation with GSH. Table 1 shows disease states in which GSH deficiency has been documented. Viral, bacterial, and fungal infections, malnutrition, chronic and acute alcohol consumption, diabetes, certain metabolic diseases, and consumption of oxidative drugs all have been shown to decrease GSH.

TABLE 1
Diseases in which GSH Deficiency Has Been
Demonstrated (WO 2005/017094, incorporated
by reference herein in its entirety).

| Classification | Disease |
|---|---|
| Hepatic Function | Acetaminophen toxicity |
| | Alcoholism |
| | Hepatitis |
| Renal Function | Chronic Kidney Failure |
| | Dialysis |
| | Nephrotoxicity |
| | Alpha-Amanitin poisoning |
| Cardiovascular | Angina |
| | Arteriosclerosis/Cardiac Risk |
| | Myocardiac Infarction |
| | Cardiomyopathy |
| Endocrine | Diabetes |
| Pulmonary | Bronchopulmonary |
| | Acute Respiratory Distress Syndrome |
| | (ARDS) |
| | Fibrosing Alveolitis |
| | Chronic Asthma |
| | Chronic Bronchitis/Chronic |
| | Obstructive Pulmonary Disease |
| | (COPD) |
| | Cystic Fibrosis |
| | Pulmonary Fibrosis |
| | Smoking |
| | Lung Cancer |
| Critical Care | Intensive Care |
| | Sepsis/Septic Shock |
| | Malnutrition |
| | Epilepsy |
| Infection | HIV |
| | Helicobacter pylori |
| | Influenza |
| | Malaria |
| Gastrointestinal | Inflammatory Bowel Disease |
| | Barrett's Esophagus |
| | Liver Disease |
| | Liver Transplantation |
| | Colon Cancer |
| Optic | Blepharitis |
| | Cataract |
| | Eale's Disease |
| Skin | Psoriasis |
| | Photodermatitis |
| Immune system | Rheumatoid Arthritis |
| | Common Variable Immunodeficiency |
| Urogenital | Prostate |
| | Urinary |
| Muscular | Exercise |
| Toxic Agents | Arsenic Poisoning |
| | Other Chemicals and Medications |
| Perinatal | Preeclampsia |
| | Neonates |
| Metabolism | Phenylketonuria |

Glutathione reductase (NADPH), a flavoprotein enzyme of the oxidoreductase class, is essential for the maintenance of cellular glutathione in its reduced form (Carlberg and Mannervick, J. Biol. Chem. 250: 5475-80 (1975)). It catalyzes the reduction of oxidized glutathione (GSSG) to reduced glutathione (GSH) in the presence of NADPH and maintains a high intracellular GSH/GSSG ratio of about 500:1 in red blood cells.

Synthesis of GSH requires cysteine, a conditionally essential amino acid that must be obtained from dietary sources or by conversion of dietary methionine via the cystathionase pathway in humans. If the supply of cysteine is adequate, normal GSH levels are maintained. But GSH depletion occurs if supplies of cysteine are inadequate to maintain GSH homeostasis in the face of increased GSH consumption. Acute GSH depletion causes severe—often fatal—oxidative and/or alkylation injury, and chronic or slow arising GSH deficiency due to administration of GSH-depleting drugs, such as acetaminophen, or to diseases and conditions that deplete GSH, can be similarly debilitating.

Replenishment of GSH requires an exogenous thiol supply, which usually is acquired by ingestion of cysteine or methionine in protein or other form. It also can be acquired by ingestion of NAC, a cysteine prodrug that is administered as the standard treatment for GSH deficiency. When administered orally or intravenously, NAC is rapidly converted to cysteine, which is then converted to GSH in the liver and elsewhere by highly regulated conversion mechanisms that maintain optimal levels of reduced GSH as long as sufficient cysteine is available for the purpose.

Cysteine is necessary to replenish hepatocellular GSH. Glutathione, glutathione monoethyl ester, and L-2-oxothiazolidine-4-carboxylate (procysteine/OTC) have been used effectively in some studies to replenish GSH. In addition, dietary methionine and S-adenosylmethionine are an effective source of cysteine.

Although various forms of cysteine and its precursors have been used as nutritional and therapeutic sources of cysteine, N-acetylcysteine (NAC) is the most widely used and extensively studied. NAC is about 10 times more stable than cysteine and much more soluble than the stable cysteine disulfide, cystine.

N-acetylcysteine (NAC) is an orally bioavailable prodrug of cysteine, which is well known for its role as an antidote against acetaminophen overdose by maintaining or restoring hepatic concentrations of glutathione via replenishing cysteine. Systemically, after NAC is biotransformed to cysteine, the latter is converted to cystine (a dimeric amino acid formed by the oxidation of two cysteine residues), a substrate for the glutamate-cystine antiporter. This antiporter allows for the uptake of cystine, which causes the reverse transport of glutamate into the extracellular space. The availability of cystine decreases the activity of the antiporter, hence reducing the transport of glutamate into the extracellular space leading to a stimulation of the inhibitory metabotropic glutamate receptors (Grant, J. et al., 2009, Arch Gen Psychiatry, 66:756-763).

Besides NAC's scavenger function, it is well-known that NAC promotes cellular glutathione production, and thus reduces, or even prevents, oxidant mediated damage. Indeed, treatment with NAC provides beneficial effects in a number of respiratory, cardiovascular, endocrine, infectious, and other disease settings as described in WO 05/017094, which is incorporated by reference herein. For example, rapid administration of NAC is the standard of care for preventing hepatic injury in acetaminophen overdose. NAC administered intravenously in dogs has been shown to protect against pulmonary oxygen toxicity and against ischemic and reperfusion damage (Gillissen, A., and Nowak, A., Respir. Med. 92: 609-23, 613 (1998)). NAC also has anti-inflammatory properties. Decreased levels of GSH are known to be associated with increased pain and fever while increased GSH levels are known to be associated with decreased pain and fever. Consistent with this inverse relationship between GSH levels and signs of inflammation (pain and/or fever), decreasing GSH renders cells more sensitive to the effects of cytokines (e.g., IL-1, IL-6, and TNF) that increase inflammation, pain and fever. Administration of N-acetylcysteine (NAC), which acts primarily to restore GSH, is known to decrease levels of IL-1, IL-6, and TNF and to reduce fever and pain (Haddad, J. et al., Molec. Immunol., 42: 987-1014, 2005; Peristeris, P. et al., Cell Immunol., 140(2): 3909, 1992).

In addition, studies have suggested that chronic oxidative stress in cystic fibrosis patients severely affects the deformability of blood neutrophils circulating in lung capillaries, thereby increases their recruitment to the lung and causes inflammation in the lungs. Systemic targeting of neutrophils in cystic fibrosis patients using high oral dosages of N-acetylcysteine have been shown to significantly reduce lung inflammation and improve lung function in the cystic fibrosis patients. (US 2007/0049641, incorporated herein by reference in its entirely). NAC treatment also has been shown to decrease NF-κB activation, which in turn decreases neutrophilic inflammation in the lung (Haddad, J. et al., Molec. Immunol., 42: 987-1014, 2005; Peristeris, P. et al., Cell Immunol., 140(2): 3909, 1992).

### 4. Involvement of Glutamatergic Dysfunction and Excessive Oxidative Stress in Autism

The existence of a glutamatergic dysfunction (Rubenstein, J and Merzeenich, M., Genes Brain Behav., 2:255-267 (2003)) and excessive oxidative stress (Kern, J. and Jones, A., J Toxicol Environ Health B Crit Rev. 9:485-499 (2006)) in autism has been proposed.

An increased ratio of excitation/inhibition in sensory, mnemonic, social, and emotional systems has been proposed as a model underlying at least some forms of autism (Rubenstein, J. and Merzenich, M., Genes Brain Behav., 2:255-267 (2003)). This hypothesis is supported by neuropathologic and neurobiologic evidence of alterations of glutamatergic transmission. Post-mortem investigations have reported increases in expression of the mRNA of several genes associated with glutamatergic pathways including excitatory amino acid transporter 1 (EAAT1) and glutamate receptor AMPA type 1, two members of the glutamate system (Purcell, A. et al., Neurology, 57: 1618-1628 (2001)). Genetic studies have also reported a link between the glutamatergic system and autism. Single nucleotide polymorphisms in the glutamate receptor 6 gene were found to be more prevalent in individuals with autism compared to typically developing controls (Jamain, S. et al., Mol Psychiatry, 7:302-310 (2002)). A susceptibility mutation in the metabotropic glutamate receptor 8 gene was also reported to be associated with autism (Serajee, F. et al, J. Med. Genet., 40:e42 (2003)). Glutamic acid decarboxylase, an enzyme that catalyzes the decarboxylation of glutamate to GABA, has also been reported to be reduced in parietal and cerebellar cortices of individuals with autism (Fatemi, S. et al., Bio Psychiatry, 52:805-810 (2002)). One study has shown a decreased density of GABA-A receptors in the anterior cingulate cortex of adults with autism (Oblak, A. et al., Autism Res. 2:205-219 (2009)).

While alterations of glutamate levels have been reported in all studies of individuals with autism, the direction (i.e., elevation or depression) of these alterations has not been consistent. Glutamate elevation has been detected in the cerebrospinal fluid in children with Rett's disorder (Hamberger, A. et al., Neuropediatrics, 23:212-213 (1992); Riikonen, R, Journal of Child Neurology, 18:693-697 (2003)) as well as in the plasma of children with autism (Moreno-Fuenmayor, H. et al., Invest Clin., 37:113-128 (1996)). In a more recent investigation, serum levels of glutamate in adult individuals with autism were found to be significantly higher than those of normal controls, and glutamate levels correlated positively with the social scores of the Autism Diagnostic Interview-Revised (ADI-R) (Shinohe, A. et al., Prog Neuropsychopharmacol Biol Psychiatry, 30:1472-1477 (2006)). In contrast, decreased levels of glutamate and GABA were found in the platelets of children with autism when compared with age-matched healthy controls (Rolf et al., Acta Psychiatr Scand., 87:312-316 (1993)).

Previous studies have suggested that redox imbalance may contribute to the neuronal insult and dysfunction seen in autism (James, S. et al., FASEB J., 23: 2374-2383 (2009)). The potential involvement of redox stress in the pathogenesis of autism has been suggested by neuropathologic (Kern, J. and Jones, A., J. Toxicol Environ Health B Crit Rev, 9:485-499 (2006)), genetic (James, S. et al., Am J Med Genet B Neuropsychiatr Genet., 141B:947-956 (2006)), and clinical studies (James, S. et al., FASEB J., 23: 2374-2383 (2009)). A decreased number of Purkinje cells (Kern, J. and Jones, A., J. Toxicol Environ Health B Crit Rev, 9:485-499 (2006)), a type of neurons in the cerebellum with selective vulnerability to toxicants, appears to be one of the most consistent neurobiological findings in autism. Furthermore, defects in neuronal migration and synaptic pruning, which were recently suggested by neuropathologic studies, might be, at least partially, related to a redox imbalance. Differences in allele frequency and/or significant gene interaction were found for relevant genes encoding glutathione-S-transferase, a key enzyme that detoxifies pro-oxidative compounds by coupling them to body's main antioxidant molecule, the tripeptide glutathione or GSH (James, S. et al., Am J Med Genet B Neuropsychiatr Genet., 141B:947-956 (2006)). Several investigations have shown decreased levels of systemic antioxidant enzymes, such as erythrocyte GSH peroxidase and superoxide dismutase (Yorbick, O. et al., Prostaglandins Leukot Essent Fatty Acids., 67:341-343 (2002)), decreased cellular and mitochondrial GSH (James, S. et al., FASEB J., 23:2374-2383 (2009)), and decreased plasma S-adenosyl-L-homocysteine (SAH) and S-adenosyl-L-methionine (SAM), intermediates in the synthesis of cysteine (James, S. et al., Am J Med Genet B Neuropsychiatr Genet., 141B:947-956 (2006)), which is a key component of GSH.

While an imbalance in the excitatory/inhibitory systems with abnormalities in the glutamatergic pathways and redox stress in the brain have been implicated in the pathophysiology of autism, there have been no definitive studies reporting positive therapeutic effects of N-acetylcysteine in the treatment of behavioral deficit in patients with autism spectrum disorder.

The described invention provides evidence that N-acetylcysteine (NAC), a glutamatergic modulator and an antioxidant known to replete GSH, is effective in the treatment of irritability and associated behavioral deficits in patients with autism.

### SUMMARY OF THE INVENTION

According to one aspect, the described invention provides a method for treating a behavioral deficit in a subject with autism spectrum disorder characterized by glutamatergic dysfunction and redox imbalance, the method comprising: (a) administering to the subject a pharmaceutical composition comprising a therapeutic amount of N-acetylcysteine, a derivative of N-acetylcysteine, or a pharmaceutically acceptable salt of N-acetylcysteine, wherein the therapeutic amount is from about 900 mg per day to about 2,700 mg per day, and wherein the therapeutic amount is effective to treat the behavioral deficit in the subject.

According to one embodiment of the method, the behavioral deficit is irritability and stereotypic or repetitive behavior. According to another embodiment, the autism spectrum disorder comprises autism, Asperger syndrome, or a pervasive developmental disorder. According to another embodiment, the N-acetylcysteine derivative comprises at least one functional group selected from the group consisting of aliphatic, aromatic, heterocyclic radical, epoxide, and arene oxide. According to another embodiment, the pharmaceutical composition further comprises a carrier. According to another embodiment, the pharmaceutical composition is a tablet. According to another embodiment, the pharmaceutical composition is an effervescent tablet. According to another embodiment, each dose of the pharmaceutical composition is individually wrapped to avoid oxidation. According to another embodiment, the composition is administered orally. According to another embodiment, administering comprises parental, intravenous, intratracheal, intramuscular, or intraperitoneal administration. According to another embodiment, the patient is administered orally about 900 mg of N-acetylcysteine, the derivative of N-acetylcysteine, or the pharmaceutically acceptable salt of N-acetylcysteine each time, three times a day. According to another embodiment, the composition is administered 900 mg per day for four weeks. According to another embodiment, the composition is administered 900 mg twice daily for four weeks. According to another embodiment, the composition is administered 900 mg three times daily for four weeks. According to another embodiment, the method further comprises monitoring a behavioral measure of the subject at a plurality of time points during treatment, relative to the measure of the behavior of the subject prior to treatment, wherein the behavioral measure comprises a primary behavioral outcome measure and a secondary behavioral outcome measure. According to another embodiment, the behavioral measure is assessed by at least one primary behavioral outcome measure consisting of Aberrant Behavior Checklist (ABC) irritability subscale, and Dosage Record and Treatment Emergent Symptom Scale (DOTES). According to another embodiment, the behavioral measure is assessed by at least one secondary behavioral outcome measure consisting of Clinical Global Impression (CGI), ABC-Stereotypy subscale, Repetitive Behavior Scale (RBS), and Social responsiveness scale (SRS). According to another embodiment, the behavioral measure is assessed before treatment, at 4 weeks, at 8 weeks, or at 12 weeks. According to another embodiment, the pharmaceutical composition comprises at least one additional therapeutic agent. According to another embodiment, the at least one additional therapeutic agent is selected from the group consisting of antipsychotic agent, an antibiotic agent, an antiviral agent, and anti-inflammatory agent, an antipyretic agent, an analgesic agent, and an anti-proliferative agent. According to another embodiment, the at least one additional therapeutic agent depletes glutathione (GSH) levels in the subject. According to another embodiment, at least one additional therapeutic agent is administered before the administration of the pharmaceutical composition. According to another embodiment, the at least one additional therapeutic agent is selected from the group consisting of antipsychotic agent, an antibiotic agent, an antiviral agent, and anti-inflammatory agent, an antipyretic agent, an analgesic agent, and an anti-proliferative agent. According to another embodiment, the at least one additional therapeutic agent depletes glutathione (GSH) levels in the subject. According to another embodiment, at least one additional therapeutic agent is administered after the administration of the pharmaceutical composition. According to another embodiment, the at least one additional therapeutic agent is selected from the group consisting of antipsychotic agent, an antibiotic agent, an antiviral agent, and anti-inflammatory agent, an antipyretic agent, an analgesic agent, and an anti-proliferative agent. According to another embodiment, the at least one additional therapeutic agent depletes glutathione (GSH) levels in the subject.

### BRIEF DESCRIPTION OF THE DRAWINGS

FIG. 1 shows patient flow diagram for N-aceyticysteine versus placebo in the treatment of children with autism.

FIG. 2 shows significant behavioral improvements with NAC treatment for the primary outcome measures; Panel (a): ABC-Irritability (F(3,75)=5.25, p=0.002) with improvement being observed in week 4 (t(25)=3.94, p=0.001) and continuing through week 8 (425)=2.87, p=0.008) and Week 12 (t(25)=3.24, p=0.004); Panel (b): ABC-total (F(3,75)=3.11, p=0.031) with significant effects beginning in week 4 (425)=3.12, p=0.005) and remaining through week 8 (425)=2.54, p=0.017) and week 12 (425)=2.73, p=0.012). Asterix denote significant (p<0.05) group differences at that time point.

### DETAILED DESCRIPTION OF THE INVENTION

### Glossary

The term “active” refers to the ingredient, component or constituent of the compositions of the described invention responsible for the intended therapeutic effect.

The term “administer” as used herein means to give or to apply. The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “analog” as used herein refers to a compound having a structure similar to another, but differing from it, for example, has one or more atoms, functional groups, or substructure.

The term “autism spectrum disorder” as used herein refers to a group of developmental disabilities that can cause significant social, communication and behavioral challenges. Examples of autistic spectrum disorder include, but are not limited to, autistic disorder (classic autism), Asperger syndrome, and pervasive developmental disorder (PSD; atypcial autism).

The term “Asperger syndrome” as used herein refers to an autism spectrum disorder, which is milder than autism but shares some of its symptoms, such as problems with language and communication, and repetitive or restrictive patterns of thoughts and behavior. An obsessive interest in a single subject is one of the major symptom of Asperger syndrome.

The term “pervasive developmental disorder” as used herein refers to a group of disorders characterized by delays in the development of socialization and communication skills. Symptoms may include problems with using and understanding language, difficulty relating to people or objects, difficulty with changes in routine or familiar surroundings, and repetitive body movements or behavior patterns.

The term “behavior” as used herein refers to the response of a system or an organism to various inputs. The behavioral functions of the brain include the processing of sensory information, the programming of motor and emotional responses, and the storing of information (memory). (Kandel, et al., Principals of Neural Science, 4^{th} Ed. (McGraw Hill, 2000), pp. 25-27). To produce a behavior, each participating sensory and motor nerve cell sequentially generates four different signals at different sites within the cell: an input signal, a trigger signal, a conducting signal, and an output signal. Generally, each nerve cell can be envisioned as comprising four functional components or regions: a local input (receptive) component, a trigger (summing or integrative) component, a long-range conducting (signaling) component, and an output (secretory) component. These functional components generate the four types of signals. Behaviors often have physical dimensions that can be measured, for example, (1) frequency (i.e., number of times a behavior occurs), (2) duration (i.e., time from which a behavior begins until it ends), and (3) intensity (physical force involved in the behavior).

The term “behavioral deficit” as used herein refers to a desirable target behavior that is seeking to be decreased or increased in frequency, duration, and intensity. For example, the term “behavioral deficit” includes, but is not limited to, an impairment in irritability, lethargy, social withdrawal, a stereotyped behavior, a self-injurious behavior, a compulsive behavior, a routine behavior, a sameness behavior, a restricted behavior, a repetitive behavior, hyperactivity, inappropriate speech, and a combination thereof.

The term “cognitive behavior” as used herein refers to a behavior influenced by thoughts and feeling.

The term “compulsive behavior” as used herein refers to a behavior of performing an act persistently and repetitively without it leading to reward or pleasure. The act is usually a small, circumscribed behavior, almost ritualistic.

The term “hyperactivity” or “hyperactive behavior” as used herein refers to a group of characteristic behaviors, including, but not limited to, constant activity, being easily distracted, impulsiveness, inability to concentrate, and aggressiveness.

The term “irritability” as used herein refers to a state of extreme sensitivity to stimulation of any kind as well as mood swings, aggression, agitation, temper outbursts, and self-injurious behaviors.

The term “lethargy” as used herein refers to feelings of tiredness, fatigue, or lack of energy.

The term “restricted behavior” refers to a behavior limited in focus, interest, or activity.

The term “repetitive behavior” as used herein refers to physical or verbal behaviors that a person engages in repeatedly. Common repetitive behaviors include, but are not limited to, head banging, thumb sucking, and rocking.

The term “social withdrawal” or “social isolation” is characterized by a lack of contact with other people in normal daily living, e.g., in the work place, with friends, and in social activities.

The term “stereotyped behavior” or “stereotypic behavior” as used herein refers to a relatively invariant mode of behavior elicited or determined by a particular situation. The stereotyped behavior may be verbal, postural, or expressive.

The terms “self-injurious behavior”, “deliberate self injury”, “self mutilation”, “self-harm” and “self-inflicted violence” as used herein refer to a deliberate harm to one's body resulting in tissue damage, without a conscious intent to die.

The term “sameness behavior” as used herein refers to a behavior which resists change.

The term “blood-brain barrier” as used herein refers to a series of structures that limit the penetration and diffusion of circulating water-soluble substances into the brain and include tight junctions between endothelial cells of brain capillaries, a dense network of astrocytes, a reduced volume of extracellular milieu and efflux pumps.

The term “carrier” as used herein describes a material that does not cause significant irritation to an organism and does not abrogate the biological activity and properties of the active substance of the composition of the described invention. Carriers must be of sufficiently high purity and of sufficiently low toxicity to render them suitable for administration to the mammal being treated. The carrier can be inert, or it can possess pharmaceutical benefits. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “component” as used herein refers to a constituent part, element or ingredient.

The term “condition”, as used herein, refers to a variety of health states and is meant to include disorders or diseases caused by any underlying mechanism or disorder, injury, and the promotion of healthy tissues and organs.

The term “contact” and all its grammatical forms as used herein refers to a state or condition of touching or of immediate or local proximity

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNF-a and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “inflammatory cytokines” or “inflammatory mediators” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1-beta (IL-1β, interleukin-4 (IL-4), interleukin-6 (IL-6), interleukin-8 (IL-8), tumor necrosis factor-alpha (TNF-a), interferon-gamma (IF-γ), and interleukin-12 (IL-12).

Among the pro-inflammatory mediators, IL-1, IL-6, and TNF-α are known to activate hepatocytes in an acute phase response to synthesize acute-phase proteins that activate complement. Complement is a system of plasma proteins that interact with pathogens to mark them for destruction by phagocytes. Complement proteins can be activated directly by pathogens or indirectly by pathogen-bound antibody, leading to a cascade of reactions that occurs on the surface of pathogens and generates active components with various effector functions. IL-1, IL-6, and TNF-a also activate bone marrow endothelium to mobilize neutrophils, and function as endogenous pyrogens, raising body temperature, which helps eliminating infections from the body. A major effect of the cytokines is to act on the hypothalamus, altering the body's temperature regulation, and on muscle and fat cells, stimulating the catabolism of the muscle and fat cells to elevate body temperature. At elevated temperatures, bacterial and viral replication are decreased, while the adaptive immune system operates more efficiently.

The term “tumor necrosis factor” as used herein refers to a cytokine made by white blood cells in response to an antigen or infection, which induce necrosis (death) of tumor cells and possesses a wide range of pro-inflammatory actions. Tumor necrosis factor also is a multifunctional cytokine with effects on lipid metabolism, coagulation, insulin resistance, and the function of endothelial cells lining blood vessels.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include, interleukin-1 (IL-1), interleukin-10 (IL-1β), interleukin-6 (IL-6), interleukin-8 (IL-8), and interleukin-12 (IL-12).

The term “derivative” as used herein means a compound that may be produced from another compound of similar structure in one or more steps. A “derivative” or “derivatives” of a compound retains at least a degree of the desired function of the compound. Accordingly, an alternate term for “derivative” may be “functional derivative.”

The derivatives of N-acetylcysteine, for example, contain one or more functional groups (e.g., aliphatic, aromatic, heterocyclic radicals, epoxides, and/or arene oxides) incorporated into N-acetylcysteine. According to another embodiment, the derivatives of N-acetylcysteine disclosed herein also comprise “prodrugs” of N-acetylcysteine, which are either active in the prodrug form or are cleaved in vivo to the parent active compound. According to another embodiment, the derivatives of N-acetylcysteine also include any pharmaceutically acceptable salt, ester, solvate, hydrate or any other compound, which, upon administration to the recipient, is capable of providing (directly or indirectly) N-acetylcysteine.

The term “disease” or “disorder”, as used herein, refers to an impairment of health or a condition of abnormal functioning.

The term “drug” as used herein refers to a therapeutic agent or any substance, other than food, used in the prevention, diagnosis, alleviation, treatment, or cure of disease.

The term “hydrophilic” as used herein refers to a material or substance having an affinity for polar substances, such as water. The term “lipophilic” as used herein refers to preferring or possessing an affinity for a non-polar environment compared to a polar or aqueous environment.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. For example, enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with a target molecule and change it chemically, for example, by modifying key amino acid residues needed for enzymatic activity. In contrast, reversible inhibitors bind non-covalently and produce different types of inhibition depending on whether these inhibitors bind the target itself, a complex formed by the target and another substance that binds to the target, or both. Inhibitors often are evaluated by their specificity and potency.

The term “macrophage” as used herein refers to a type of white blood cell that surrounds and kills microorganisms, removes dead cells, and stimulates the action of other immune system cells. After digesting a pathogen, a macrophage presents an antigen (a molecule, most often a protein found on the surface of the pathogen, used by the immune system for identification) of the pathogen to the corresponding helper T cell. The presentation is done by integrating it into the cell membrane and displaying it attached to an MHC class II molecule, indicating to other white blood cells that the macrophage is not a pathogen, despite having antigens on its surface. Eventually, the antigen presentation results in the production of antibodies that attach to the antigens of pathogens, making them easier for macrophages to adhere to with their cell membrane and phagocytose.

The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “prevent” as used herein refers to the keeping, hindering or averting of an event, act or action from happening, occurring, or arising.

The term “prodrug” as used herein means a substance or derivative which is in an inactive form and which is converted to an active form by biological conversion following administration to a subject.

The term “recombinant” as used herein refers to a substance produced by genetic engineering.

The term “redox stress” or “redox imbalance” as used herein refers to disequilibrium between oxidants and antioxidants in the body, which leads to accumulation of reactive oxygen species (ROS).

The term “reduced” or “to reduce” as used herein refer to a diminution, a decrease, an attenuation or abatement of the degree, intensity, extent, size, amount, density or number.

The term “similar” is used interchangeably with the terms analogous, comparable, or resembling, meaning having traits or characteristics in common.

The term “susceptible” as used herein refers to a member of a population at risk.

The terms “subject” or “individual” or “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The phrase “subject in need of such treatment” as used herein refers to a patient who displays symptoms of autism or an autism spectrum disorder or who will otherwise benefit from the described treatment, including, without limitation, one who (i) will receive treatment with the composition of the invention; (ii) is receiving the composition of the invention; or (iii) has received the composition of the invention. In some other embodiments, the phrase “subject in need of such treatment” also is used to refer to a patient who (i) will suffer from autism or an autism spectrum disorder; (ii) is suffering from autism or an autism spectrum disorder; or (iii) has suffered from autism or an autism spectrum disorder. In some other embodiments, the phrase “subject in need of such treatment” also is used to refer to a patient who (i) will be administered a composition of the invention; (ii) is receiving a composition of the invention; or (iii) has received a composition of the invention, unless the context and usage of the phrase indicates otherwise.

The term “symptom” as used herein refers to a sign or an indication of disorder or disease, especially when experienced by an individual as a change from normal function, sensation, or appearance.

The term “syndrome,” as used herein, refers to a pattern of symptoms indicative of some disease or condition.

The term “therapeutic agent” as used herein refers to a drug, molecule, nucleic acid, protein, composition or other substance that provides a therapeutic effect. The terms “therapeutic agent” and “active agent” are used interchangeably. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “therapeutic amount” or an “amount effective” of one or more of the active agents is an amount that is sufficient to provide the intended benefit of treatment. However, dosage levels are based on a variety of factors, including the type of injury, the age, weight, sex, medical condition of the patient, the severity of the condition, the route of administration, and the particular active agent employed. The amount of the active in the compositions of the present invention, which will be effective in the treatment of a particular autism disorder or condition will depend on the nature of the disorder or condition, and can be determined by standard clinical techniques. See, for example, Goodman and Gilman; The Physician's Desk Reference, Medical Economics Company, Inc., Oradell, N.J., (1995); and Drug Facts and Comparisons, Facts and Comparisons, Inc., St. Louis, Mo., (1993). The precise dose to be employed in the formulation will also depend on the route of administration, and the seriousness of the autism disease or disorder, and should be decided according to the judgment of the practitioner and each patient's circumstances.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned, which does not cause substantial toxicity and yet is effective to treat the particular subject. The effective amount for any particular application may vary depending on such factors as the disease or condition being treated, the particular therapeutic agent(s) being administered, the size of the subject, or the severity of the disease or condition. One of ordinary skill in the art may determine empirically the effective amount of a particular therapeutic agent(s) without necessitating undue experimentation. It generally is preferred that a maximum dose be used, that is, the highest safe dose according to some medical judgment. The terms “dose” and “dosage” are used interchangeably herein.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

Methods for Treating a Behavioral Deficit in a Subject with Autism

According to one aspect, the described invention provides a method for treating a behavioral deficit in a subject with autism spectrum disorder characterized by glutamatergic dysfunction and redox imbalance, wherein the method comprises administering a pharmaceutical composition comprising a therapeutic amount of N-acetylcysteine, a derivative of N-acetylcysteine, or a pharmaceutically acceptable salt thereof, wherein the therapeutic amount is from about 900 mg per day to about 2,700 mg per day, and wherein the therapeutic amount is effective to improve or reduce the behavioral deficit.

According to one embodiment of the method, the behavioral deficit includes, but is not limited to irritability and stereotypic, or repetitive, behavior.

According to another embodiment, the autism spectrum disorder includes, but is not limited to, autism, Asperger syndrome, and pervasive developmental disorder.

Over-the-counter NAC can be variably produced and packaged. Because the production and packaging methods generally do not guard against oxidation, the NAC can be significantly contaminated with bioactive oxidation products. These may be particularly important in view of data indicating that the oxidized form of NAC has effects counter to those reported for NAC and is bioactive at doses roughly 10-100 fold less than NAC (see Samstrand et al J. Pharmacol. Exp. Ther. 288: 1174-84 (1999)).

The distribution of the oxidation states of NAC as a thiol and disulfide depends on the oxidation/reduction potential. The half-cell potential obtained for the NAC thiol/disulfide pair is about +63 mV, indicative of its strong reducing activity among natural compounds (see Noszal et al. J. Med. Chem. 43:2176-2182 (2000)).

NAC is easily oxidized when exposed to air and an open bottle of capsules is very vulnerable to oxidation. Therefore, in some embodiments of the invention, the preparation and storage of the formulation is performed in such a way that the reduced form of NAC is the primary form administered to the patient. According to some such embodiments, NAC-containing formulations are maintained in solid form.

According to some embodiments, NAC is formulated as an effervescent tablet dosage form. Effervescent tablets allow for an even distribution of NAC concentration and create a balanced buffered solution for easy absorption. According to some such embodiments, in order to protect each NAC effervescent tablet from degradation and oxidation, each dose of the NAC composition is vacuum-wrapped in four-layer foil packaging. According to another embodiment, each dose of the NAC composition is vacuum-wrapped in four-layer paper packaging. According to another embodiment, each dose of the NAC composition is vacuum-wrapped in four-layer plastic packaging. According to some such embodiments, the tablet contains about 900 mg of NAC. According to another embodiment, the tablet contains about 800 mg of NAC. According to another embodiment, the tablet contains about 700 mg of NAC. According to another embodiment, the tablet contains about 600 mg of NAC.

According to some other embodiments, NAC is formulated as a coated tablet, including, but not limited to, a sugar-coated tablet, a gelatin-coated tablet, a film-coated tablet, an enteric-coated tablet, a double-layer tablet, and a multi-layer tablet.

When in solution, NAC containing formulations are preferably stored in a brown bottle that is vacuum sealed. Storage in cool dark environments is also preferred.

The determination of reduced and oxidized species present in a sample may be determined by various methods known in the art, for example with capillary electrophoresis, HPLC, etc. as described by Chassaing et al. J Chromatogr B Biomed Sci Appl 735(2):219-27 (1999).

The N-acetylcysteine or the N-acetylcysteine derivative in the compositions are delivered in therapeutically amounts. According to another embodiment, the N-acetylcysteine derivative comprises at least one functional group selected from the group consisting of aliphatic, aromatic, heterocyclic radical, epoxide, and arene oxide.

According to some embodiments, the therapeutic amount of N-acetylcysteine or a derivative thereof is about 1.8 grams per day (“g/d”) to about 6.9 g/d (i.e., a minimum of about: 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 g/d and a maximum of about: 6.0, 5.8, 5.8, 5.7, 5.6, 5.5, 5.4, 5.3, 5.2, 5.1, 5.0, 4.9, 4.8, 4.7, 4.6, 4.5, 4.4., 4.3, 4.2, 4.1, 4.0, 2.9, 2.8, 2.7, 2.6, 2.5, 2.4, 2.3, 2.2, 2.1, 2.0, 1.9, or 1.8 g/d), not to exceed about 70 mg per kg per day (“mg/kg/d”).

According to some embodiments, administering occurs orally. According to some such embodiments, the therapeutic amount of N-acetylcysteine or a derivative thereof is from about 200 mg to about 20,000 mg per dosage unit when formulated for oral administration. According to another embodiment, the therapeutic amount of N-acetylcysteine or a derivative thereof is about 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 11000 mg, 12000 mg, 13000 mg, 14000 mg, 15000 mg, 16000 mg, 17000 mg, 18000 mg, 19000 mg, or 20,000 mg when formulated for oral administration. According to another embodiment, the therapeutic amount of N-acetylcysteine or a derivative thereof when formulated for parenteral administration is no more than about: 20000 mg, 19000 mg, 18000 mg, 17000 mg, 16000 mg, 15000 mg, 14000 mg, 13000 mg, 12000 mg, 11000 mg, 10000 mg, 9500 mg, 9000 mg, 8500 mg, 8000 mg, 7500 mg, 7000 mg, 6500 mg, 6000 mg, 5500 mg, 5000 mg, 4500 mg, 4000 mg, 3500 mg, 3000 mg, 2500 mg, 2000 mg, 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, 500 mg, 400 mg, 300 mg, or 200 mg.

Solid dosage forms for oral administration may include capsules, tablets, pills, powders, granules, and gels. In such solid dosage forms, the active compounds may be admixed with at least one inert diluent such as sucrose, lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

According to another embodiment, administering occurs intratracheally.

According to another embodiment, administering occurs parenterally. According to some such embodiments, the therapeutic amount of N-acetylcysteine or a derivative thereof when formulated for parenteral administration is at least about: 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 11000 mg, 12000 mg, 13000 mg, 14000 mg, 15000 mg, 16000 mg, 17000 mg, 18000 mg, 19000 mg, or 20,000 mg. According to another embodiment, the therapeutic amount of N-acetylcysteine or a derivative thereof when formulated for parenteral administration is no more than about: 20000 mg, 19000 mg, 18000 mg, 17000 mg, 16000 mg, 15000 mg, 14000 mg, 13000 mg, 12000 mg, 11000 mg, 10000 mg, 9500 mg, 9000 mg, 8500 mg, 8000 mg, 7500 mg, 7000 mg, 6500 mg, 6000 mg, 5500 mg, 5000 mg, 4500 mg, 4000 mg, 3500 mg, 3000 mg, 2500 mg, 2000 mg, 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, 500 mg, 400 mg, 300 mg, or 200 mg.

According to another embodiment, administering occurs intravenously. According to another embodiment, administering occurs intramuscularly. According to another embodiment, administering occurs intraperitoneally, intradermally, subcutaneously, subdurally, intracerebrally, intrathecally, or topically.

According to another embodiment, the composition is administered 1 to 4 times a day. According to another embodiment, the composition is administered once a day, twice a day, three times a day or four times a day. According to another embodiment, the composition is administered in a daily treatment cycle for a period of time, or is administered in a cycle of every other day, every third day, every fourth day, every firth day, every 6th day or once a week for a period of time, the period of time being from one week to several months, for example, 1, 2, 3, or 4 weeks, or 1, 2, 3, 4, 5, or 6 months.

According to another embodiment, the composition is administered at a dose of 900 mg per day. According to another embodiment, the composition is administered at a dose of 900 mg twice daily. According to another embodiment, the composition is administered at a dose of 900 mg three times daily.

According to another embodiment, the composition is administered at a dose of 900 mg per day for four weeks. According to another embodiment, the composition is administered at a dose of 900 mg twice daily for four weeks. According to another embodiment, the composition is administered at a dose of 900 mg three times daily for four weeks. According to another embodiment, the composition is administered at a dose of 900 mg per day for the first four weeks, then 900 mg twice daily for four weeks, and 900 mg three times daily for four weeks.

The formulations of therapeutic agent(s) may be administered in pharmaceutically acceptable solutions, which may routinely contain pharmaceutically acceptable concentrations of salt, buffering agents, preservatives, compatible carriers, adjuvants, and optionally other therapeutic ingredients.

In certain instances, it may be advantageous to administer NAC or a functional derivative of NAC in combination with at least one additional pharmaceutical (or therapeutic) agent (e.g., antipsychotic typically used in autism patients to control aberrant behaviors, a selective serotonin re-uptake inhibitor typically used in autism patients to control aberrant behaviors, an antibiotic agent, an antiviral agent, an anti-inflammatory agent, an antipyretic agent, an analgesic agent, an anti-proliferative agent). For example, a compound of the present invention may be administered simultaneously with, or before or after, one or more other therapeutic agent(s). Alternatively, the compound of the present invention may be administered separately, by the same or different route of administration, or together in the same pharmaceutical composition as the other agent(s). Such combinations may offer significant advantages, including additive activity or synergistic activity, in therapy. According to some embodiments, the at least one additional pharmaceutical (or therapeutic) agent is capable of depleting GSH levels in the subject. According to some such embodiments, NAC or a functional derivative of NAC replenishes GSH levels in the subject that are depleted by the additional pharmaceutical (or therapeutic) agent that is capable of depleting GSH levels.

In the combination therapies of the invention, the compound of the present invention and the other therapeutic agent may be manufactured and/or formulated by the same or different manufacturers. Moreover, the compound of the present invention and the other therapeutic (or pharmaceutical agent) may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; or (iii) in the patient themselves, e.g. during sequential administration of the compound of the invention and the other therapeutic agent.

According to another embodiment, the composition may be prepared in a solid form (including granules, powders or suppositories) or in a liquid form (e.g., solutions, suspensions, or emulsions).

According to another embodiment, the carrier of the composition of the described invention includes a release agent, such as sustained release or delayed release carrier. In such embodiments, the carrier can be any material capable of sustained or delayed release of N-acetylcysteine or a derivative thereof to provide a more efficient administration, e.g., resulting in less frequent and/or decreased dosage of N-acetylcysteine or a derivative thereof, improve ease of handling, and extend or delay effects on diseases, disorders, conditions, syndromes, and the like, being treated, prevented or promoted. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

According to another embodiment, the N-acetylcysteine or the N-acetylcysteine derivative of the described invention may be applied in a variety of solutions. A suitable formulation is sterile, dissolves sufficient amounts of the N-acetylcysteine or a derivative thereof, and is not harmful for the proposed application. For example, the compositions of the described invention may be formulated as aqueous suspensions wherein the active ingredient(s) is (are) in admixture with excipients suitable for the manufacture of aqueous suspensions.

Such excipients include, without limitation, suspending agents (e.g., sodium carboxymethylcellulose, methylcellulose, hydroxy-propylmethylcellulose, sodium alginate, polyvinylpyrrolidone, gum tragacanth, and gum acacia), dispersing or wetting agents including, a naturally-occurring phosphatide (e.g., lecithin), or condensation products of an alkylene oxide with fatty acids (e.g., polyoxyethylene stearate), or condensation products of ethylene oxide with long chain aliphatic alcohols (e.g., heptadecaethyl-eneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate).

Compositions of the described invention also may be formulated as oily suspensions by suspending the active ingredient in a vegetable oil (e.g., arachis oil, olive oil, sesame oil or coconut oil) or in a mineral oil (e.g., liquid paraffin). The oily suspensions may contain a thickening agent (e.g., beeswax, hard paraffin or cetyl alcohol).

Compositions of the described invention also may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients also may be present.

Compositions of the described invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Thus, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate.

The formulations may be presented conveniently in unit dosage form and may be prepared by any of the methods well known in the art of pharmacy. All methods include the step of bringing into association a therapeutic agent(s), or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The pharmaceutical agent, a pharmaceutically acceptable salt, or a functional derivative of the described invention may be mixed with other active materials that do not impair the desired action, or with materials that supplement the desired action. Solutions or suspensions used for parenteral, intradermal, subcutaneous, subdural, intracerebral, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules (or ampules), disposable syringes or multiple dose vials made of glass or plastic. Administered intravenously, particular carriers are physiological saline or phosphate buffered saline (PBS).

According to another embodiment, the method further comprises monitoring a behavioral measure of the subject receiving the composition, relative to the behavioral measure of the subject prior to treatment. The behavioral measure includes, but is not limited to, primary behavioral outcome measures and secondary behavioral outcome measures.

Primary behavioral outcome measures, include, for example, but are not limited to, (a) the Aberrant Behavior Checklist (ABC) irritability subscale; and (b) Dosage Record and Treatment Emergent Symptom Scale (DOTES), which provides information on the presence, frequency and severity of side effects (Guy, W., ECDEU Assessment Manual for Psychopharmacology, Rockville, Md., U.S. Department of Health, Education, and Welfare (1976)). Secondary behavioral outcome measures include, but are not limited to, (a) the Clinical Global Impression (CGI); (b) ABC-Stereotypy subscale; (c) Repetitive Behavior Scale (RBS); and (d) Social responsiveness scale (SRS).

The Aberrant Behavior Checklist (ABC) is a standardized scale comprising 58 items for assessing problem behavior in subjects with mental retardation and developmental disabilities (Aman, M. et al., Am J Ment Defic. 89:492-502 (1985)). The checklist is empirically derived from ratings on approximately 1000 subjects, and the items resolve into five subscales: irritability, lethargy/social withdrawal, stereotypic behavior, hyperactivity, and inappropriate speech. High scores indicate more severe behavioral symptoms.

The Clinical Global Impression (CGI) rating scales are commonly used measures of symptom severity, treatment response and the efficacy of treatments in treatment studies of patients with mental disorders (Guy, W., 1976, ECDEU Assessment Manual for Psychopharmacology, Rockville, Md., U.S. Department of Health, Education, and Welfare). The Clinical Global Impression-Severity scale (CGI-S) is a 7-point scale that requires the clinician to rate the severity of the patient's illness at the time of assessment, relative to the clinician's past experience with patients who have the same diagnosis. Considering total clinical experience, a patient is assessed on severity of mental illness at the time of rating 1=normal, not at all ill; 2=borderline mentally ill; 3=mildly ill; 4=moderately ill; 5=markedly ill; 6=severely ill; or 7=extremely ill.

The Repetitive Behavior Scale-Revised (RBS-R) (Bodfish et al., The Repetitive Behavior Scale: A test manual (1998)) is an empirically derived clinical rating scale for measuring the presence and severity of a variety of forms of restricted, repetitive behavior that are characteristic of individuals with autism. The RBS-R consists of 6 subscales: stereotyped behavior, self-injurious behavior, compulsive behavior, routine behavior, sameness behavior, and restricted behavior. The scale provides an overall raw score for severity of repetitive behaviors and separate measures of severity for each subtype of repetitive behavior. High scores indicate more severe behavioral symptoms.

The Social Responsiveness Scale (SRS) (Constantino, J. et al., J Dev Behav Pediatr, 21:2-11 (2000); Constantino, J. et al., J Autism Dev Disord., 3:427-433 (2003)) is a norm-referenced, 65-item parent report questionnaire developed to measure social behaviors, including social awareness, social information processing, reciprocal social communication, and social anxiety, in both clinical and non-clinical populations. It is designed for use with children ages 4 through 18, and more recently, a special version of the SRS has been developed for preschoolers (Pine, E. et al., Autism, 10:344-352 (2006)). The SRS items measure the Autistic Spectrum Disorder (ASD) symptoms in the domains of social awareness, social information processing, reciprocal social communication, social anxiety/avoidance, and stereotypic behavior/restricted interests. Each item is scored from 1 (not true) to 4 (almost always true). Scores are obtained for five treatment subscales: Social Awareness (e.g., “Is aware of what others are thinking or feeling”), Social Cognition (e.g., “Doesn't recognize when others are trying to take advantage of him or her”), Social Communication (e.g., “Avoids eye contact or has unusual eye contact”), Social Motivation (e.g., “Would rather be alone than with others”), and Autistic Mannerisms (e.g., “Has an unusually narrow range of interests”). The SRS summary score was continuously distributed within each group and minimally correlated with Intelligent Quotient (IQ). High scores indicate more severe behavioral symptoms.

According to another embodiment, the behavioral measure is assessed prior to a treatment. According to another embodiment, the behavioral measure is assessed immediately following administration of the composition of the described invention. According to another embodiment, the behavioral measure is assessed at one day, two days, three days, four days, five days, six days, one week, two weeks, three weeks, four weeks, five weeks, six weeks, seven weeks, eight weeks, nine weeks, ten weeks, eleven weeks, or twelve weeks after administration of the composition of the described invention. According to another embodiment, the behavioral measure is assessed at five months, six months, seven months, eight months, nine months, ten months, eleven months, or one year after administration of the composition.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods and Materials

### Study Design

A 12-week, double-blind randomized, placebo-controlled study of NAC in children with autism was conducted in the Autism & Developmental Disabilities Clinic in the Division of Child & Adolescent Psychiatry at Stanford University Medical Center. The recruitment period started in March 2009 and ended in September 2010. Subjects with and without mental retardation were included. After obtaining informed consent, subjects were screened and inclusion and exclusion criteria were assessed. No changes in eligibility criteria were applied throughout the study. This investigation was approved by the institutional review board at Stanford University School of Medicine. An investigational new drug application (IND#100905) was filed with the Food and Drug Administration, and the study was registered in National Institutes of Health at ClinicalTrials.gov (identifier number NCT00627705).

### Inclusion and Exclusion Criteria

Inclusion criteria included were: (a) outpatients between 3 and 12 years of age; (b) males and females who were physically healthy; (c) diagnosis of autism based on DSM-IV-TR criteria, the Autism Diagnostic Interview-Revised (ADI-R) and/or Autism Diagnostic Observation Schedule (ADOS), and expert clinical evaluation; (d) Clinical Global Impressions (CGI) Severity rating of greater than 4; (e) care provider who could reliably bring subject to clinic visits, could provide trustworthy ratings, and interacted with subject on a regular basis; (f) stable concomitant medications and biomedical treatments for at least 2 weeks; and (g) no planned changes in psychosocial interventions during the trial.

Exclusion criteria included were: (a) DSM-IV diagnosis of schizophrenia, schizoaffective disorder, or psychotic disorder not otherwise specified; (b) prior adequate trial of NAC; (c) active medical problems: unstable seizures, significant physical illness; (d) pregnancy or sexually active females. In addition, (e) subjects taking antioxidant agents and GSH prodrugs were excluded from the study except when they had been off these compounds for at least 4 weeks.

### Interventions

After the screening phase, baseline measures were obtained from subjects continuing to meet inclusion and exclusion criteria. Subjects were then randomized to either placebo or active based on age and gender. As NAC is a nutritional supplement, the quality control is predictably variable and therefore purity is not as stringent as prescription medications. Therefore, in the present study, the stability of the compound was ascertained and the integrity of the active agent was protected by packaging each NAC doses in individual pockets. The active compound and matching placebo were provided by BioAdvantex Pharma, Inc. (Mississauga, Ontario, Canada). The term “matching placebo” as used herein refers to an inactive substance or preparation used as a control in a clinical to determine the effectiveness of the active agent, NAC. Subjects randomized to the active drug were initiated at the dose of 900 mg every day for the first 4 weeks, then 900 mg twice daily for 4 weeks and 900 mg three times daily for 4 weeks. (This dose selection was based on previously published studies for other psychiatric conditions (Grant, J. et al., Arch Gen Psychiatry, 66:468-475 (2009); Berk, M. et al., Biol Psychiatry, 64:361-368 (2008); Berk, M. et al., Bio Psychiatry, 64:468-475 (2008)) and the previous experience of our group in studies of children with cystic fibrosis). Alternatively, subjects were initiated at the dose of 900 mg per day, 1,800 mg per day, or 2,700 mg per day of NAC. Alternatively, the subjects were initiated at the dose ranging from 900 mg per day to 2,700 mg per day of NAC.

If a subject could not tolerate a specific dose, s/he would be maintained at the highest tolerated dose. Subjects were evaluated at baseline, week 4, week 8 and week 12. The Aberrant Behavioral Checklist (ABC) (Aman, M. et al., Am J Ment Defic. 89:492-502 (1985)), the Clinical Global Impressions (CGI) (Guy, W., ECDEU Assessment Manual for Psychopharmacology, Rockville, Md., U.S. Department of Health, Education, and Welfare (1976)), and the Dosage Record and Treatment Emergent Symptom Scale (DOTES) (Guy, W., 1976, ECDEU Assessment Manual for Psychopharmacology, Rockville, Md., U.S. Department of Health, Education, and Welfare) were obtained in each visit. Additional secondary measures were obtained at baseline and at week 12 and included the social responsiveness scale (SRS) (Constantino, J. et al., J. Dev Behav Pediatr., 21:2-11 (2000); Constantino, J. et al., J Autism Dev Disord., 33:427-433 (2003)), and the repetitive behavior scale-revised (RBS-R) (Bodfish, J. et al., The Repetitive Behavior Scale: A test manual (1998)).

### Statistical Analyses

To examine the primary hypothesis that active treatment with NAC would decrease irritability and overall negative behavior associated with autism, we computed mixed effects regression models with ABC-Irritability and Total scores as the primary dependent variables in separate analyses. Treatment Group (2 levels: NAC vs. placebo) and Time (4-levels: baseline, week 4, week 8, and week 12) and their interaction were covariates. The interaction of Treatment Group X Time directly tests the hypothesis by examining whether treatment groups showed a different pattern of change in symptoms across study time points. Significant interactions were followed by independent samples t-tests at each post-baseline time point (Week 4, Week 8, and Week 12) to determine the time of onset of treatment effects. Additional mixed effect regression models were computed using other ABC sub-scales, SRS Total and sub-scales, and RBS Total scores as dependent variables. These exploratory analyses examined specific treatment effects for lethargy, stereotypy/repetitive behavior, hyperactivity, inappropriate speech, and social communication behavior. CGI-S and CGI-I scores were also examined in the same manner. All models were fit using an autoregressive covariance structure.

The same set of analyses was also performed examining only participants who completed the study. A conservative last observation carried forward (LOCF) analysis was also computed for all randomized participants who had any follow-up data. Type 1 error rate of 0.05 was used for the primary and all exploratory analyses. Multiple comparison correction was not performed for exploratory measures as this is a small initial study and the purpose of the exploratory analyses was to better understand the specificity of the treatment effect.

### Example 1

### Study Population

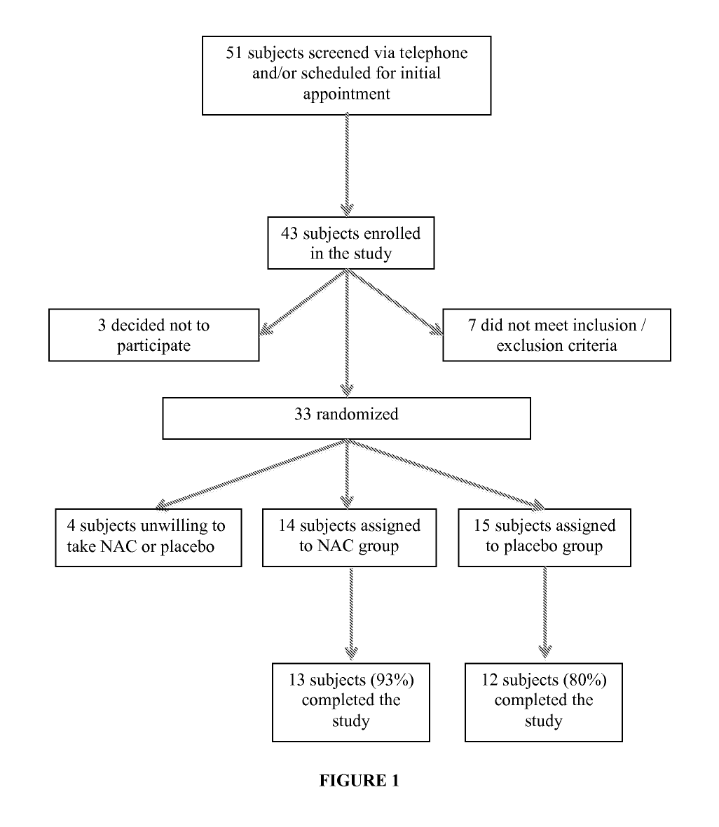

FIG. 1 shows the patient disposition throughout the study. Fifty-one potential subjects inquired about the study. Forty-three of them signed an informed consent form. Seven did not meet the inclusion/exclusion criteria and 3 decided not to participate in the study before baseline measures were obtained. Thirty-three subjects representing 31 males and 2 females aged 3.2 to 10.7 years were randomized in the study. Fifteen were randomized to receive the composition containing NAC and eighteen were randomized to the placebo group. Four were unwilling to take the compound because of its taste (1 active and 3 placebo), so analyses were completed using data from the remaining subjects (NAC N=14, placebo N=15). There were no differences between the placebo group and the active group on any of the demographic and clinical baseline measures (Table 2). Mean age of subjects randomized in the NAC and placebo groups was 7.0±2.1 and 7.2±2.2 years, respectively. Twenty-five subjects (NAC N=13, placebo N=12) completed the study. Fourteen subjects were on at least on one psychotropic medication with three being on more than one. The most common prescribed classes of medications were second generation antipsychotics and selective serotonin re-uptake inhibitors. Four subjects were missing the ADI-R and ADOS.

TABLE 2
Baseline Comparison of Participants With Autism
Assigned to Receive N-Acetylcysteine or Placebo

| | | | | | p |
|---|---|---|---|---|---|
| | Placebo | | NAC | | value |
| # in group | 18 | | 15 | | |
| Male/Female | 18/0 | | 13/2 | | .199 |
| Age (years) | 7.2 (2.2) | [3.2-10.7] | 7.0 (2.1) | [4.4-10.4] | .802 |
| ABC | 63.6 (28.5) | [28-123] | 69.7 (24.7) | [15-104] | .499 |
| total score | | | | | |
| ABC | 14.8 (9.6) | [5-41] | 16.9 (7.9) | [1-27] | .510 |
| irritability | | | | | |
| score | | | | | |
| CGI severity | 5.3 (0.8) | [3-6] | 5.1 (0.7) | [4-6] | .498 |
| score | | | | | |
| SRS total | 104.7 (28.1) | [48-158] | 111.9 (28.3) | [64-150] | .478 |
| RBS total | 38.2 (24.0) | [16-115] | 33.1 (16.2) | [8-66] | .490 |

Abbreviations: ABC, Aberrant Behavioral Checklist; CGI, Clinical Global Impression; SRS, Social Responsiveness Scale; RBS, Repetitive Behavior Scale. P value for sex based on Fisher's exact test; P values for all other variables based on independent samples t-test.

### Example 2

### Power for Primary Outcome Measures

Power to detect a N-acetylcysteine (NAC) treatment effect was examined using the observed sample size (29 total; 15 placebo, 14 NAC) and a four time point repeated measures ANOVA model. This model is typically conservative relative to the mixed effects regression models implemented in the present study which incorporate all available data and explicitly model relationships between time points. For this analysis, the correlation between repeated measures at baseline, week 4, week 8, and week 12 was conservatively estimated to be r=0.50. The observed correlations actually tended to be larger (r=0.43-0.86). Results indicated excellent power (0.89) to detect a medium effect size (f=0.25, d=0.50) for NAC treatment (a=0.05, two-tailed). Power to detect smaller treatment effects (f=0.10, d=0.20) was much weaker (0.20).

### Example 3

### Behavioral Outcomes

TABLE 3
Treatment Responses of Participants with Autism Assigned
to Receive N-Acetylcysteine (NAC) or Placebo

| | Mean (SD) [Range] | | | | | | |
|---|---|---|---|---|---|---|---|
| | Baseline | | Week 12 | | | | |
| | Placebo | NAC | Placebo | NAC | | | |
| | (n = 18) | (n = 15) | (n = 15) | (n = 14) | F | p | Cohen's d |
| ABC total | 63.6 (28.5) | 69.7 (24.7) | 53.9 (24.4) | 38.7 (24.1) | 4.06 | .010 | .63 |
| | [28-123] | [15-104] | [14-114] | [3-85] | | | |
| ABC irritability | 14.8 (9.6) | 16.9 (7.9) | 13.1 (9.9) | 7.2 (5.7) | 6.80 | <.001 | .72 |
| | [5-41] | [1-27] | [4-41] | [0-18] | | | |
| ABC lethargy | 12.1 (7.8) | 15.2 (9.5) | 8.3 (7.7) | 11.0 (9.4) | 1.93 | .134 | −.32 |
| | [1-24] | [2-31] | [1-23] | [0-32] | | | |
| ABC stereotypy | 8.9 (6.5) | 9.1 (5.5) | 8.0 (7.0) | 5.6 (5.7) | 2.21 | .096 | .37 |
| | [0-21] | [2-21] | [1-18] | [0-19] | | | |
| ABC | 23.8 (9.3) | 23.4 (9.0) | 21.0 (11.5) | 12.4 (11.4) | 1.97 | .130 | .75 |
| hyperactivity | [8-37] | [6-37] | [3-31] | [1-27] | | | |
| ABC | 4.1 (3.7) | 4.9 (3.2) | 3.6 (3.6) | 2.5 (2.6) | 1.25 | .297 | .35 |
| inappropriate | [0-11] | [0-11] | [0-11] | [0-7] | | | |
| speech | | | | | | | |
| CGI severity | 5.3 (0.8) | 5.1 (0.7) | 4.9 (0.9) | 4.5 (0.8) | 1.73 | .170 | .47 |
| | [3-6] | [4-6] | [3-6] | [3-6] | | | |
| CGI | — | — | 3.2 (0.9) | 2.9 (1.1) | 0.81 | .449 | .30 |
| improvement | | | [2-5] | [2-6] | | | |
| SRS total | 104.7 (28.1) | 111.9 (28.3) | 98.5 (37.8) | 93.8 (26.7) | 2.36 | .141 | .14 |
| | [48-158] | [64-150] | [35-148] | [44-135] | | | |
| RBS total | 38.2 (24.0) | 33.1 (16.2) | 33.4 (24.2) | 22.3 (12.0) | 6.80 | .015 | .57 |
| | [16-115] | [8-66] | [6-105] | [2-41] | | | |

Abbreviations: ABC, Aberrant Behavioral Checklist; CGI, Clinical Global Impression SRS, Social Responsiveness Scale; RBS, Repetitive Behavior Scale. Means and standard deviations are derived from all observed data at the respective time points. F-values are derived from the interaction of Participant Group (NAC vs. Placebo) and Time (Week) in mixed effects regression models. Cohen's d was computed based on the standardized mean difference between groups at Week 12. For ABC and CGI-Severity, regression estimated degrees of freedom were 3.66 or 3.67. For CGI-Improvement degrees of freedom were 2.49. For SRS total and RBS total, degrees of freedom were 1.22 and 1.24.

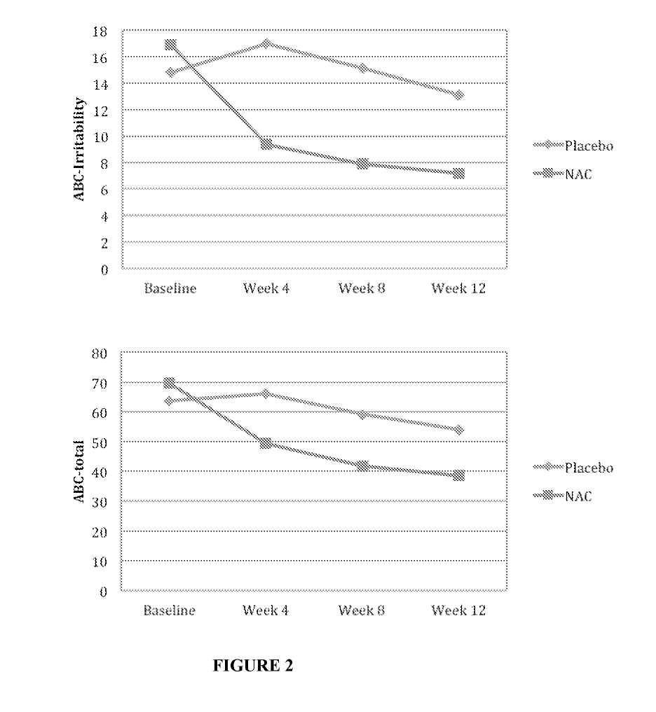

FIG. 2 presents results for the primary outcome measures, ABC-Irritability and ABC-Total, across the four study time points. When examining all randomized participants, NAC treatment significantly improved irritability (F(3,66)=6.80, p<0.001), with effects beginning in week 4 (425)=3.94, p=0.001; FIG. 2a) and continuing through week 8 (425)=2.87, p=0.008) and Week 12 (t(25)=3.24, p=0.004). The same pattern was present for ABC-Total Scores (F(3,66)=4.06, p=0.010; FIG. 2b) with significant effects beginning in week 4 (t(25)=3.12, p=0.005) and remaining through week 8 (t(25)=2.54, p=0.017) and week 12 (t(25)=2.73, p=0.012). NAC treatment resulted in marginally significant improvement in stereotypic/repetitive behavior on the ABC (ABC-Stereotypy F(3,67)=2.21, p=0.096) and significant improvement on the RBS-Total (F(1,24)=6.80, p=0.015). Additionally, NAC treatment did not significantly influence SRS-Total raw scores (F(1,20)=2.36, p=0.141), but there were significant improvements in SRS-Social Cognition (F(1,20)=4.99, p=0.037 and SRS-Autism Mannerisms (F(1,20)=4.56, p=0.045) sub-scales. However, the improvements in SRS-Social Cognition appear to be due to greater baseline impairment in the NAC group followed by regression to the mean rather than a true treatment effect. There were no significant treatment effects for any other SRS subscale or for ABC hyperactivity, lethargy, and inappropriate speech subscales (all p>0.100), although interestingly there was a large reduction (d=0.75) in hyperactivity at Week 12 that was less striking in earlier weeks. There were no significant differences in the pattern of global severity (F(3,66)=1.70, p=0.170) or improvement (F(2,49)=0.81, p=0.449), possibly due to the limited scaling and sensitivity of the CGI-S and CGI-I. Finally, additional analyses were conducted for participants who completed the study (NAC N=13, and placebo N=12). A similar pattern of results emerged with one notable exceptions: SRS-Total scores were significantly improved by NAC treatment (p=0.039).

### Example 4

### Safety Evaluation

Minimal side effects were observed with the exception of one subject in the active group who experienced worsening of baseline agitation and irritability requiring early termination which led to symptom resolution. This participant exhibited the same behavioral worsening 6 weeks after being terminated from the study, which led to a medical evaluation that revealed severe constipation. The following adverse events (AEs) were more frequently reported in the NAC group than the placebo group, but differences were not statistically different (all p>0.100): depressed affect (1 vs. 0), akathisia (1 vs. 0), constipation (3 vs. 2), nausea/vomiting (6 vs. 3), increased appetite (2 vs. 0), diarrhea (3 vs. 1). The following AEs were reported more in the placebo group than the NAC group: excitement/agitation (3 vs. 2), increased motor activity (3 vs. 2), nasal congestion (6 vs. 4), decreased appetite (3 vs. 2), tremor (1 vs. 0), sweating (1 vs. 0), syncope/dizziness (1 vs. 0), increased salivation (2 vs. 0). One subject in each group reported insomnia and stomachache.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

What is claimed is:

1. A method for treating a behavioral deficit in a subject with autism spectrum disorder characterized by glutamatergic dysfunction and redox imbalance, the method comprising:
(a) administering to the subject a pharmaceutical composition comprising a therapeutic amount of N-acetylcysteine, a derivative of N-acetylcysteine, or a pharmaceutically acceptable salt of N-acetylcysteine, wherein the therapeutic amount is from about 900 mg per day to about 2,700 mg per day, and wherein the therapeutic amount is effective to treat the behavioral deficit in the subject.

2. The method according to claim 1, wherein the behavioral deficit is irritability and stereotypic or repetitive behavior.

3. The method according to claim 1, wherein the autism spectrum disorder comprises autism, Asperger syndrome, or a pervasive developmental disorder.

4. The method according to claim 1, wherein the N-acetylcysteine derivative comprises at least one functional group selected from the group consisting of aliphatic, aromatic, heterocyclic radical, epoxide, and arene oxide.

5. The method according to claim 1, wherein the pharmaceutical composition further comprises a carrier.

6. The method according to claim 1, wherein the pharmaceutical composition is a tablet.

7. The method according to claim 6, wherein the pharmaceutical composition is an effervescent tablet.

8. The method according to claim 6, wherein each dose of the pharmaceutical composition is individually wrapped to avoid oxidation.

9. The method according to claim 1, wherein the composition is administered orally.

10. The method according to claim 1, wherein administering comprises parental, intravenous, intratracheal, intramuscular, or intraperitoneal administration.

11. The method according to claim 1, wherein the patient is administered orally about 900 mg of N-acetylcysteine, the derivative of N-acetylcysteine, or the pharmaceutically acceptable salt of N-acetylcysteine each time, three times a day.

12. The method according to claim 9, wherein the composition is administered 900 mg per day for four weeks.

13. The method according to claim 9, wherein the composition is administered 900 mg twice daily for four weeks.

14. The method according to claim 9, wherein the composition is administered 900 mg three times daily for four weeks.

15. The method according to claim 1, wherein the method further comprises monitoring a behavioral measure of the subject at a plurality of time points during treatment, relative to the measure of the behavior of the subject prior to treatment, wherein the behavioral measure comprises a primary behavioral outcome measure and a secondary behavioral outcome measure.

16. The method according to claim 15, wherein the primary behavioral outcome measure comprises an Aberrant Behavior Checklist (ABC) irritability subscale, a Dosage Record and Treatment Emergent Symptom Scale (DOTES), or a combination thereof.

17. The method according to claim 15, wherein the secondary behavioral outcome measure comprises Clinical Global Impression (CGI), ABC-Stereotypy subscale, Repetitive Behavior Scale (RBS), Social responsiveness scale (SRS), or a combination thereof.

18. The method according to claim 15, wherein the behavioral measure is assessed before treatment, at 4 weeks, at 8 weeks, or at 12 weeks.

19. The method according to claim 1, wherein the pharmaceutical composition comprises at least one additional therapeutic agent.

20. The method according to claim 19, wherein the at least one additional therapeutic agent is selected from the group consisting of an antipsychotic agent, an antibiotic agent, an antiviral agent, an anti-inflammatory agent, an antipyretic agent, an analgesic agent, and an anti-proliferative agent.

21. The method according to claim 19, wherein the at least one additional therapeutic agent is capable of depleting glutathione (GSH) levels in the subject.

22. The method according to claim 1, wherein at least one additional therapeutic agent is administered before the administration of the pharmaceutical composition.

23. The method according to claim 22, wherein the at least one additional therapeutic agent is selected from the group consisting of an antipsychotic agent, an antibiotic agent, an antiviral agent, an anti-inflammatory agent, an antipyretic agent, an analgesic agent, and an anti-proliferative agent.

24. The method according to claim 22, wherein the at least one additional therapeutic agent is capable of depleting glutathione (GSH) levels in the subject.

25. The method according to claim 1, wherein at least one additional therapeutic agent is administered after the administration of the pharmaceutical composition.

26. The method according to claim 25, wherein the at least one additional therapeutic agent is selected from the group consisting of an antipsychotic agent, an antibiotic agent, an antiviral agent, an anti-inflammatory agent, an antipyretic agent, an analgesic agent, and an anti-proliferative agent.

27. The method according to claim 25, wherein the at least one additional therapeutic agent is capable of depleting glutathione (GSH) levels in the subject.
